(19) United States
(12) Patent Application Publication
(10) Pub. No.: US 2023/0277511 A1
(43) Pub. Date: Sep. 7, 2023

(54) RAPAMYCIN (RAPA) COMPOSITION AND PREPARATION METHOD THEREOF

(71) Applicant: Pengke YAN, Guangzhou (CN)

(72) Inventor: Pengke YAN, Guangzhou (CN)

(21) Appl. No.: 18/024,054

(22) PCT Filed: May 31, 2021
(86) PCT No.: PCT/CN2021/097356

§ 371 Date: Mar. 1, 2023

(30) Foreign Application Priority Data
Sep. 30, 2020 (CN) 202011061905.1

(51) Int. Cl. A61K 31/436; A61K 47/68; A61K 47/61; A61K 47/54; A61K 9/127; A61K 47/28; A61K 47/24; A61K 47/69

(52) U.S. Cl. CPC A61K 31/436; A61K 47/6849; A61K 47/61; A61K 47/549; A61K 9/127; A61K 47/28; A61K 47/24; A61K 47/6913; A61K 47/6911

(57) ABSTRACT

A rapamycin (RAPA) composition and a preparation method thereof are provided. The RAPA composition includes the following active ingredients in parts by weight: RAPA: 1 to 10 parts; polymer carrier: 0.5 to 20 parts; and lymphatic target: 0.1 to 1 part. The lymphatic target is at least one selected from the group consisting of sodium hyaluronate (SH), an aptamer, and an antibody. The preparation method includes: preparing an organic phase solution by adding RAPA to an organic phase solvent to obtain the organic phase solution; preparing an emulsion by adding a polymer carrier to an aqueous phase solvent and adding the organic phase solution dropwise to the aqueous phase solvent to obtain the emulsion; and conducting homogenization by adding a lymphatic target to the emulsion, mixing, and homogenizing to obtain the RAPA composition. The RAPA composition can target a lymphatic system to treat atherosclerosis (AS) and related cardiovascular diseases (CVDs).

### CROSS REFERENCE TO THE RELATED APPLICATIONS

This application is the national phase entry of International Application No. PCT/CN2021/097356, filed on May 31, 2021, which is based upon and claims priority to Chinese Patent Application No. 202011061905.1, filed on Sep. 30, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBRZBC070_Sequence Listing_20230322.txt, created on 03/22/2023, and is 3,101 bytes in size.

### TECHNICAL FIELD

The present disclosure relates to a rapamycin (RAPA) composition and a preparation method thereof and belongs to the technical field of medicine.

### BACKGROUND

Atherosclerosis (AS) is a chronic inflammatory disease and is an abnormal response of a blood vessel wall to various injuries, but the action mechanism of AS has always been unclear. Early studies have shown that there are a large number of lymphatic vessels around an atherosclerotic blood vessel, but the relationship between the two has always been unclear. Recent studies have shown that lymphatic vessels are not only involved in the initiation and regression of arterial inflammation but also play a positive role in reverse cholesterol transport (RCT). Lymphatic vessels accompany blood vessels in tissues and carry out functions such as returning tissue fluid, immune cells, and lipoproteins. The excretion of cholesterol plaques also depends on the transport of lymphatic vessels. Studies have confirmed that, with the slowdown of lymphatic drainage, plasma substances accumulate locally on a blood vessel wall. Lymphatic vessels in the outer membrane of the main artery constitute reticular tissue at marginal areas of the medial and outer membranes of the main artery. The drainage of lymphatic vessels plays an important role in the discharge of infiltrated glia and macromolecules from an arterial wall, and the accumulation of the glia and macromolecules is considered as a key element in the occurrence of an atherosclerotic lesion.

RAPA is a macrolide antibiotic and is mainly used for the treatment of immune rejection in transplantation. In recent years, more and more studies and medications have shown that RAPA exhibits a prominent effect in the treatment of rare lymphatic malformation diseases. It has been found in clinical treatment that there are successful clinical cases in the treatment of kaposiform lymphangiomatosis (KLA), lymphangioleiomyomatosis (LAM), large-area capillary-lymphangio-venous malformation, and lymphatic hamartomatosis with RAPA. However, there has been no report on the studies of RAPA in the treatment of AS.

### SUMMARY

To overcome the deficiencies of the prior art, a first objective of the present disclosure is to provide a RAPA composition. The RAPA composition can target a lymphatic system to treat AS and related cardiovascular diseases (CVDs) through the lymphatic system.

A second objective of the present disclosure is to provide a preparation method of the RAPA composition.

The first objective of the present disclosure may be achieved through the following technical solutions: A RAPA composition is provided, including the following active ingredients in parts by weight:

| | |
|---|---|
| RAPA | 1 to 10 parts; |
| polymer carrier | 0.5 to 20 parts; and |
| lymphatic target | 0.1 to 1 part. |

The lymphatic target is at least one selected from the group consisting of sodium hyaluronate (SH), an aptamer, and an antibody.

The specific binding capacity of the aptamer to lymphatic endothelial cells (LECs) is higher than or equal to 50%.

The antibody is at least one selected from the group consisting of a lymphatic vessel endothelial hyaluronic acid receptor antibody and a human recombinant Prox protein antibody.

Further, the polymer carrier is at least one selected from the group consisting of polyethylene glycol (PEG), polylactic-co-glycolic acid (PLGA), polyethyleneoxide (PEO), polyvinylpyrrolidone (PVP), polypropylene (PP), polyamino acid (PAA), polysorbate, a polyoxyethylene fatty acid, a methoxypolyethylene glycol (mPEG) block copolymer, and methoxypolyethylene glycol-polylactide (mPEG-PLA).

Further, the SH has a molecular weight of 5,000 to 20,000.

Further, the aptamer has 20 bp to 120 bp of bases.

Further, the overlap rate between a nucleotide sequence of the aptamer and any one of the sequences shown in SEQ ID NOs: 1-10 is higher than or equal to 50%.

Further, the RAPA composition includes a phospholipid; the phospholipid is at least one selected from the group consisting of lecithin, cephalin, phosphatidylserine, phosphatidylglycerol (PG), phosphatidylinositol (PI), sphingomyelin, diphosphatidylglycerol (DPG), dipalmitoylphosphatidylcholine (DPPC), dioleoylphosphatidylethanolamine (DOPE), and distearoylphosphatidylethanolamine (DSPE).

Further, the phospholipid is added in 1 to 20 parts by weight.

Further, the RAPA composition includes cholesterol.

Further, the cholesterol is added in 0.1 to 1 part by weight.

The second objective of the present disclosure may be achieved through the following technical solutions: A preparation method of a RAPA composition is provided, including:

preparing an organic phase solution by adding RAPA to an organic phase solvent to obtain the organic phase solution;

preparing an emulsion by adding a polymer carrier to an aqueous phase solvent, adding the organic phase solution dropwise to the aqueous phase solvent, and mixing to obtain the emulsion; and

conducting homogenization by adding a lymphatic target to the emulsion, mixing, and homogenizing to obtain the RAPA composition.

Further, in the preparation of the organic phase solution, the organic phase solvent is at least one selected from the group consisting of absolute ethanol, dichloromethane (DCM), tertiary butyl alcohol (TBA), acetone, and methanol.

Further, in the preparation of the emulsion, the mixing is achieved by stirring for 30 min to 3 h at room temperature and a stirring speed of 300 rpm to 1,200 rpm.

Further, the preparation method further includes conducting lyophilization by adding a lyophilization protective agent to the RAPA composition, filtering a resulting mixture through a microporous filter membrane for sterilization, and lyophilizing.

Further, in the lyophilization, the lyophilization protective agent is added at an amount of 5 to 20 g per 100 mL of the RAPA composition.

Compared with the prior art, the present disclosure has the following beneficial effects:
1. The RAPA composition of the present disclosure can target a lymphatic system to improve the accumulation of RAPA in the lymphatic system, has a half-life of 50 h or more in the blood, and can directly reach a lymphatic system. The continuous administration of the RAPA composition can treat a lymph-related disease such as AS to reduce AS plaques. Therefore, the RAPA composition can treat AS and related CVDs through a lymphatic system.
2. In the preparation method of the RAPA composition of the present disclosure, a lymphatic target is used for hydrophilic surface modification, an average particle size is controlled at 50 nm to 200 nm, a drug load is 0.1% to 20%, an encapsulation rate can reach 70% or higher, and a uniform and stable particle size distribution is achieved.
3. In the preparation method of the RAPA composition of the present disclosure, a polymer carrier is used to encapsulate RAPA, a lymphatic target is embedded on the surface, and an encapsulation structure is dispersed into a nano-scale encapsulated particle through the dispersion of an organic phase solvent and an aqueous phase solvent.

### BRIEF DESCRIPTION OF THE DRAWINGS

FIG. 1 shows the appearance of the products obtained in Examples 1 to 5;

FIG. 2 shows the liposome simulation of the RAPA composition obtained in Example 4;

FIG. 3 shows a particle size distribution of a RAPA composition;

FIG. 4 is a transmission electron microscopy (TEM) image of a RAPA composition;

FIG. 5 shows cell survival curves;

FIG. 6 is a histogram of RAPA enrichment;

FIG. 7 shows an AS effect of a blank group;

FIG. 8 shows an AS effect of a control group;

FIG. 9 shows an AS effect of a drug group; and

FIG. 10 is a histogram of plaque areas.

### DETAILED DESCRIPTION OF THE EMBODIMENTS

The present disclosure is further described below in combination with the accompanying drawings and specific implementations.

A RAPA composition is provided, including the following active ingredients in parts by weight:

| | |
|---|---|
| RAPA | 1 to 10 parts; |
| polymer carrier | 0.5 to 20 parts; |
| lymphatic target | 0.1 to 1 part; |
| phospholipid | 1 to 20 parts; and |
| cholesterol | 0.1 to 1 part. |

The lymphatic target is at least one selected from the group consisting of SH, an aptamer, and an antibody.

The SH has a molecular weight of 5,000 to 20,000.

An overlap rate between a nucleotide sequence of the aptamer and any one of the sequences shown in SEQ ID NOs: 1-10 is higher than or equal to 50%, and the overlap rate may be at least 50%, at least 60%, at least 70%, at least 80%, or at least 90%. A specific binding capacity of the aptamer to LECs is higher than or equal to 50%, and the specific binding capacity is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100%.

The antibody is at least one selected from the group consisting of a lymphatic vessel endothelial hyaluronic acid receptor antibody (LYVE-1) and a human recombinant Prox protein antibody.

The lymphatic target can specifically recognize LECs, which enables the RAPA composition to target lymph.

The polymer carrier is at least one selected from the group consisting of PEG (including PEG-2000, PEG-4000, PEG-10000, and PEG-15000), PLGA, PEO, PVP, PP, PAA, polysorbate, a polyoxyethylene fatty acid, an mPEG block copolymer, and mPEG-PLA. Compared with a small-molecular-weight carrier, the polymer carrier can lead to a sustained-release formulation with a relatively long hydrophilic time, which can improve the sustained-release time of an injection prepared from RAPA in the body and prolong the half-life of RAPA. The polymer carrier can be degraded under natural physiological conditions and thus excreted through metabolism without irritation or foreign body response to the body.

The phospholipid is at least one selected from the group consisting of lecithin, cephalin, phosphatidylserine, PG, PI, sphingomyelin, DPG, DPPC, DOPE, and DSPE.

The lymphatic target is modified with the polymer, phospholipid, and cholesterol, such that the RAPA composition can be well produced.

A preparation method of a RAPA composition is provided, including:

Preparation of an organic phase solution: RAPA, a phospholipid, and cholesterol are added to 40 to 200 parts of an organic phase solvent to obtain the organic phase solution.

Preparation of an emulsion: A polymer carrier is added to an aqueous phase solvent, and the organic phase solution is added at a speed of 1 to 10 drops/min dropwise to 300 to 20,000 parts of the aqueous phase solvent. The resulting mixture is stirred for 30 min to 3 h at room temperature (25° C.) and a stirring speed of 300 rpm to 1,200 rpm to obtain the emulsion. At room temperature, the raw materials are relatively stable and the organic phase solvent can be easily volatilized.

Homogenization: A lymphatic target is added to the emulsion, and the resulting mixture is stirred for 30 min to 3 h and then homogenized 5 to 20 times under a homogenization pressure of 300 bar to 1,000 bar to obtain a RAPA composition.

Lyophilization: A lyophilization protective agent is added to the RAPA composition at an amount of 5 g to 20 g per 100 mL of the RAPA composition, and the resulting mixture is filtered through a microporous filter membrane with a pore size of 0.22 m to 0.45 m for sterilization and then lyophilized.

The organic phase solvent is at least one selected from the group consisting of absolute ethanol, DCM, TBA, acetone, and methanol. The organic phase solvent needs to have excellent solubility for the RAPA, phospholipid, cholesterol, and polymer carrier. An organic phase solution is first prepared and then slowly released into an aqueous phase solvent. The aqueous phase solvent is stirred such that a nano-scale solution is produced from the RAPA, phospholipid, cholesterol, and polymer carrier due to the physical force and the dissolution rate difference. Free RAPA is removed through filtration to obtain a RAPA composition with low hazard to the blood health.

The aqueous phase solvent is at least one selected from the group consisting of distilled water, normal saline (NS), cell culture medium, body fluid, buffer, and glucose injection. The aqueous phase solvent provides an excellent dispersion medium for the organic phase solvent. The hydrophilic polymer carrier and the dispersion of the organic phase solvent can effectively improve the dispersion of RAPA in the aqueous phase solvent, thereby producing a nano-scale particle.

The lyophilization protective agent is at least one selected from the group consisting of lactose, glucose, mannitol, and sucrose.

The RAPA composition of the present disclosure can target a lymphatic system to improve the accumulation of RAPA in the lymphatic system, has a residence time of 24 h to 48 h in the lymphatic system (resulting in a sustained release effect), a half-life of 50 h or more in the blood, and can directly reach a lymphatic system. The RAPA composition is injected at an amount of 10 μg/mL or more based on the active ingredient RAPA. After the continuous administration of the RAPA composition, AS plaques are reduced.

### Example 1

A RAPA composition was provided in Example 1, including the following active ingredients:

| | |
|---|---|
| RAPA | 120 mg |
| PEG2000-DSPE | 120 mg |
| SH (molecular weight: 8,000) | 60 mg |
| Lecithin | 960 mg |
| Cholesterol | 120 mg |

Organic phase solvent: 10 mL of absolute ethanol; and aqueous phase solvent: 15 mL of PBS+150 mL of pure water.

A preparation method of the RAPA composition was provided, including:

Preparation of an organic phase solution: RAPA, lecithin, and cholesterol were added to the organic phase solvent to obtain the organic phase solution.

Preparation of an emulsion: PEG2000-DSPE was added to the aqueous phase solvent. The organic phase solution was added at a speed of 5 drops/min dropwise to the aqueous phase solvent. The resulting mixture was stirred at a stirring speed of 500 rpm for mixing and then stirred at room temperature and a stirring speed of 600 rpm for 1 h to obtain the emulsion.

Homogenization: SH was added to the emulsion, and the resulting mixture was stirred for 1 h and then homogenized 6 times under a homogenization pressure of 600 bar to obtain a RAPA composition.

Lyophilization: Lactose (a lyophilization protective agent) was added to the RAPA composition at an amount of 10 g per 100 mL of the RAPA composition. The resulting mixture was filtered through a microporous filter membrane with a pore size of 0.22 m for sterilization and then lyophilized.

### Example 2

A RAPA composition was provided in Example 2, including the following active ingredients:

| | | |
|---|---|---|
| RAPA | 120 | mg |
| PEG2000-DSPE | 100 | mg |
| SH (molecular weight: 8,000) | 80 | mg |
| Lecithin | 1,000 | mg |
| Cholesterol | 100 | mg |

Organic phase solvent: 10 mL of absolute ethanol; and aqueous phase solvent: 15 mL of PBS+150 mL of pure water.

A preparation method of the RAPA composition was the same as that in Example 1.

### Example 3

A RAPA composition was provided in Example 3, including the following active ingredients:

| | |
|---|---|
| RAPA | 120 mg |
| PEG2000-DSPE | 80 mg |
| SH (molecular weight: 8,000) | 80 mg |
| Lecithin | 720 mg |
| Cholesterol | 80 mg |

Organic phase solvent: 10 mL of DCM; and aqueous phase solvent: 15 mL of PBS+150 mL of pure water.

A preparation method of the RAPA composition was the same as that in Example 1.

### Example 4

A RAPA composition was provided in Example 4, including the following active ingredients:

| | |
|---|---|
| RAPA | 120 mg |
| PEG2000-DSPE | 80 mg |
| SH (molecular weight: 10,000) | 80 mg |
| Lecithin | 840 mg |
| Cholesterol | 120 mg |

Organic phase solvent: 10 mL of absolute ethanol; and aqueous phase solvent: 15 mL of PBS+150 mL of pure water.

A preparation method of the RAPA composition was the same as that in Example 1.

### Example 5

A RAPA composition was provided in Example 5, including the following active ingredients:

| | |
|---|---|
| RAPA | 120 mg |
| PEG2000-DSPE | 80 mg |
| SH (molecular weight: 10,000) | 80 mg |
| Lecithin | 720 mg |
| Cholesterol | 120 mg |

Organic phase solvent: 10 mL of DCM; and aqueous phase solvent: 15 mL of PBS+150 mL of pure water.

A preparation method of the RAPA composition was the same as that in Example 1.

### Example 6

A RAPA composition was provided in Example 6, including the following active ingredients:

| | |
|---|---|
| RAPA | 120 mg |
| PEG2000-DSPE | 80 mg |
| SH (molecular weight: 10,000) | 80 mg |
| Lecithin | 960 mg |
| Cholesterol | 80 mg |

Organic phase solvent: 10 mL of absolute ethanol; and aqueous phase solvent: 15 mL of PBS+150 mL of pure water.

A preparation method of the RAPA composition was provided, including:

Preparation of an organic phase solution: RAPA, lecithin, and cholesterol were added to the organic phase solvent to obtain the organic phase solution.

Preparation of an emulsion: PEG2000-DSPE was added to the aqueous phase solvent. The organic phase solution was added at a speed of 5 drops/min dropwise to the aqueous phase solvent. The resulting mixture was stirred at a stirring speed of 450 rpm for mixing and then stirred at room temperature and a stirring speed of 600 rpm for 1 h to obtain the emulsion.

Homogenization: SH was added to the emulsion, and the resulting mixture was stirred for 1 h and then homogenized 10 times under a homogenization pressure of 500 bar to obtain a RAPA composition.

Lyophilization: Lactose (a lyophilization protective agent) was added to the RAPA composition at an amount of 10 g per 100 mL of the RAPA composition, and the resulting mixture was filtered through a microporous filter membrane with a pore size of 0.22 m for sterilization and then lyophilized.

### Example 7

A RAPA composition was provided in Example 7, including the following active ingredients:

| | |
|---|---|
| RAPA | 120 mg |
| PEG2000-DSPE | 80 mg |
| SH (molecular weight: 10,000) | 80 mg |
| Lecithin | 720 mg |
| Cholesterol | 80 mg |

Organic phase solvent: 10 mL of absolute ethanol; and aqueous phase solvent: 15 mL of PBS+150 mL of pure water.

A preparation method of the RAPA composition was the same as that in Example 6.

### Example 8

A RAPA composition was provided in Example 8, including the following active ingredients:

| | |
|---|---|
| RAPA | 120 mg |
| PEG2000-DSPE | 100 mg |
| Lymphatic vessel endothelial hyaluronic acid receptor | 60 mg |
| antibody (LYVE-1) | |
| Lecithin | 960 mg |
| Cholesterol | 120 mg |

Organic phase solvent: 10 mL of absolute ethanol; and aqueous phase solvent: 15 mL of PBS+150 mL of pure water.

A preparation method of the RAPA composition was provided, including:

Preparation of an organic phase solution: RAPA, lecithin, and cholesterol were added to the organic phase solvent to obtain the organic phase solution.

Preparation of an emulsion: PEG2000-DSPE was added to the aqueous phase solvent. The organic phase solution was added at a speed of 5 drops/min dropwise to the aqueous phase solvent. The resulting mixture was stirred at a stirring speed of 500 rpm for mixing and then stirred at room temperature and a stirring speed of 500 rpm for 1 h to obtain the emulsion.

Homogenization: The lymphatic vessel endothelial hyaluronic acid receptor antibody (LYVE-1) was added to the emulsion, and the resulting mixture was stirred for 1 h and then homogenized 10 times under a homogenization pressure of 400 bar to obtain an RAPA composition.

Lyophilization: Lactose (a lyophilization protective agent) was added to the RAPA composition at an amount of 10 g per 100 mL of the RAPA composition, and the resulting mixture was filtered through a microporous filter membrane with a pore size of 0.45 m for sterilization and then lyophilized.

### Example 9

A RAPA composition was provided in Example 9, including the following active ingredients:

| | | |
|---|---|---|
| RAPA | 120 | mg |
| PEG2000-DSPE | 120 | mg |
| Aptamer shown in SEQ ID NO: 1 | 1.6 | mg |
| Lecithin | 840 | mg |
| Cholesterol | 120 | mg |

Organic phase solvent: 10 mL of absolute ethanol; and aqueous phase solvent: 15 mL of PBS+150 mL of pure water.

A preparation method of the RAPA composition was provided, including:

Preparation of an organic phase solution: RAPA, lecithin, and cholesterol were added to the organic phase solvent to obtain the organic phase solution.

Preparation of an emulsion: PEG2000-DSPE was added to the aqueous phase solvent. The organic phase solution was added at a speed of 5 drops/min dropwise to the aqueous phase solvent. The resulting mixture was stirred at a stirring speed of 500 rpm for mixing and then stirred at room temperature and a stirring speed of 500 rpm for 1 h to obtain the emulsion.

Homogenization: The aptamer shown in SEQ ID NO: 1 was added to the emulsion, and the resulting mixture was stirred for 1 h and then homogenized 10 times under a homogenization pressure of 400 bar to obtain a RAPA composition.

Lyophilization: Lactose (a lyophilization protective agent) was added to the RAPA composition at an amount of 10 g per 100 mL of the RAPA composition, and the resulting mixture was filtered through a microporous filter membrane with a pore size of 0.22 m for sterilization and then lyophilized.

### Example 10

A RAPA composition was provided in Example 10, including the following active ingredients:

| | | |
|---|---|---|
| RAPA | 120 | mg |
| PEG2000-DSPE | 120 | mg |
| Aptamer shown in SEQ ID NO: 3 | 1.2 | mg |
| Lecithin | 960 | mg |
| Cholesterol | 84 | mg |

Organic phase solvent: 10 mL of DCM; and aqueous phase solvent: 15 mL of PBS+150 mL of pure water.

A preparation method of the RAPA composition was provided, including:

Preparation of an organic phase solution: RAPA, lecithin, and cholesterol were added to the organic phase solvent to obtain the organic phase solution.

Preparation of an emulsion: PEG2000-DSPE was added to the aqueous phase solvent. The organic phase solution was added at a speed of 5 drops/min dropwise to the aqueous phase solvent. The resulting mixture was stirred at a stirring speed of 500 rpm for mixing and then stirred at room temperature and a stirring speed of 600 rpm for 1 h to obtain the emulsion.

Homogenization: The aptamer shown in SEQ ID NO: 3 was added to the emulsion, and the resulting mixture was stirred for 1 h and then homogenized 6 times under a homogenization pressure of 600 bar to obtain a RAPA composition.

Lyophilization: Lactose (a lyophilization protective agent) was added to the RAPA composition at an amount of 10 g per 100 mL of the RAPA composition, and the resulting mixture was filtered through a microporous filter membrane with a pore size of 0.22 m for sterilization and then lyophilized.

Performance Test:
1) Each of the RAPA compositions obtained in Examples 1 to 10 was evaluated for appearance and tested for average particle size, potential, and encapsulation rate.

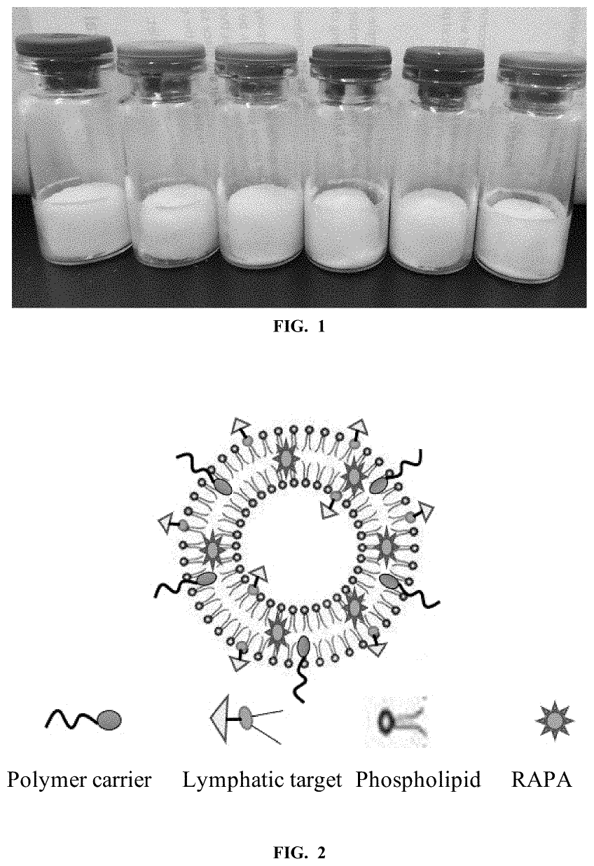

Appearance evaluation criteria: The following characteristics were desired: original volume, no collapse, no shrinkage, uniform color, no spot, and delicate texture. The appearance of the RAPA compositions of Examples 1 to 5 was shown from left to right in FIG. 1.

Average particle size: A Malvern laser particle size analyzer was used to determine the particle size and particle size distribution of nanoparticles. The particle size was determined according to the following principle: When particles are irradiated by light, light scattering and light diffraction occur, and the scattering intensity and diffraction intensity of light are both related to particle sizes and optical characteristics.

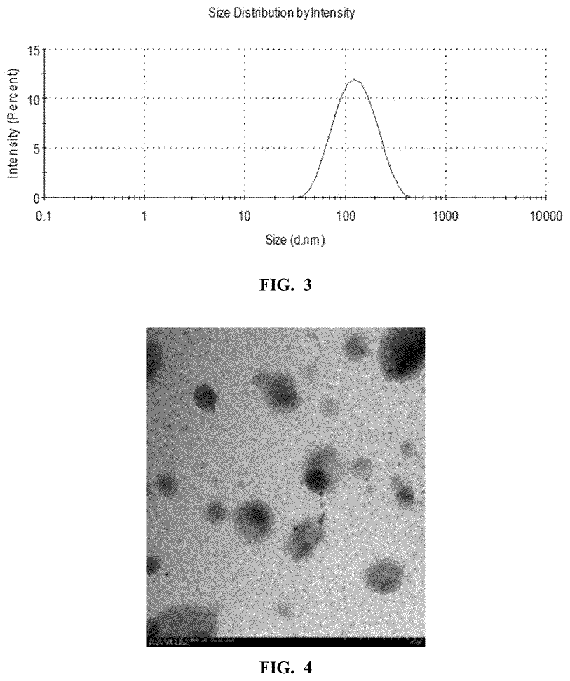

FIG. 2 shows the liposome simulation of the RAPA composition obtained in Example 4, where globular portions represent the active ingredient RAPA and linear portions represent the polymer carrier and the lymphatic target. FIG. 3 shows a particle size distribution of a RAPA composition. FIG. 4 is a TEM image of a RAPA composition.

Encapsulation rate: An encapsulation rate of 70% or higher was desired.

The total content of the drug was determined using a content determination method.

The drug content was determined by high-performance liquid chromatography (HPLC) with methanol-acetonitrile-water (in a volume ratio of 43:40:17) as a mobile phase, a flow rate of 1 mL/min, a column temperature of 40° C., and a detection wavelength of 278 nm.

The calculation formula for the encapsulation rate: encapsulation rate=encapsulated drug amount/total main drug content×10000

TABLE 1
Changes in appearance, redispersibility, encapsulation
rate, and average particle size for RAPA compositions

| | | | | Average |
|---|---|---|---|---|
| | | | | particle |
| | | | Encapsulation | size |
| Example | Appearance | Redispersibility | rate (%) | (nm) |
| 1 | No shrinkage and | Excellent | 71 | 120 |
| | no collapse | | | |
| 2 | No shrinkage and | Excellent | 80 | 116 |
| | no collapse | | | |
| 3 | No shrinkage and | Excellent | 87 | 121 |
| | no collapse | | | |
| 4 | No shrinkage and | Excellent | 72 | 105 |
| | no collapse | | | |
| 5 | No shrinkage and | Excellent | 76 | 112 |
| | no collapse | | | |
| 6 | No shrinkage and | Excellent | 77 | 102 |
| | no collapse | | | |
| 7 | No shrinkage and | Excellent | 81 | 107 |
| | no collapse | | | |
| 8 | No shrinkage and | Excellent | 84 | 113 |
| | no collapse | | | |
| 9 | No shrinkage and | Excellent | 75 | 109 |
| | no collapse | | | |
| 10 | No shrinkage and | Excellent | 73 | 93 |
| | no collapse | | | |

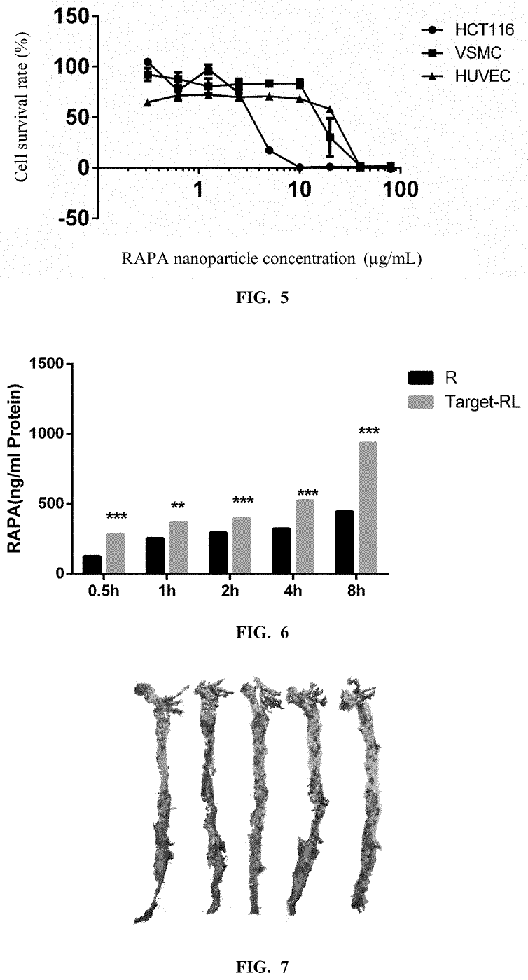

The RAPA composition obtained in the present disclosure as an encapsulation rate of 700% or higher.
2) An MTT kit method was used to conduct a cytotoxicity experiment with HCT116 cells, human vascular smooth muscle cells (HVSMCs), and human umbilical vein endothelial cells (HUVECs). The HCT116 cells were inoculated at 1×10⁴ cells/well, the HVSMCs were inoculated at 7×10³ cells/well, and the HUVECs were inoculated at 1×10⁴ cells/well into 96-well plates. The cells were cultivated in a 500 CO₂/37° C. incubator for 24 h and treated for 48 h with the RAPA composition in Example 4 at concentrations (based on the active ingredient RAPA) of 80 μg/mL, 40.00 μg/mL, 30.00 μg/mL, 20.00 μg/mL, 10.00 μg/mL, 5.00 μg/mL, 2.50 μg/mL, 1.25 μg/mL, 0.65 μg/mL, 0.3125 μg/mL, and 0 μg/mL. Results are shown in FIG. 5, and the results showed that the RAPA composition significantly inhibited the proliferation of the above cells. After the 48 h of administration, an IC₅₀ value for the HCT116 was 5 μg/mL, an IC₅₀ value for the HVSMCs was 13 μg/mL, and an IC₅₀ value for the HUVECs was 17 g/mL.
3) Lymph-targeted effect of the RAPA composition

Animal: New Zealand white rabbits each with a weight of 4 kg to 6 kg were purchased from Guangdong Medical Laboratory Animal Center.

Grouping and experimental scheme: Blank group: 1 mL of NS was injected into each rabbit through the ear vein. Control group (R): The active ingredient RAPA was diluted with NS to a desired concentration (administration amount: 0.5 mg/kg) and injected into each rabbit through the ear vein. Drug group (Target-RL): The RAPA composition in Example 4 was injected at 1.5 mg/mL into each rabbit through the ear vein. At 1 h, 2 h, 4 h, 8 h, and 24 h after injection, lymphatic fluid was collected from the rabbit, and RAPA in the lymphatic fluid was extracted to determine the RAPA content in the lymphatic fluid. According to the RAPA content, whether the RAPA composition targeted a lymphatic system was determined. Results are shown in FIG. 6, and the results showed that the RAPA composition was concentrated in the lymphatic fluid at different time points in larger quantities than the active ingredient RAPA with a significant difference, indicating that the RAPA composition has a lymph-targeted effect.
4) Therapeutic effect of the RAPA composition on AS

Animal: Apolipoprotein gene-knockout mice (ApoE−/− mice) each with a weight of 18 g to 25 g were purchased from Peking University Laboratory Animal Center.

Construction of AS model mice: The mice were raised to be 8 weeks old in an SPF-grade environment (room temperature: 23° C., relative humidity: 65%, and 12 h light-dark cycle) and then fed with a high-fat feed for 12 weeks to obtain the AS model mice, and then each of the AS model mice was administered. Blood vessels were collected and stained, and a proportion of a plaque area was calculated according to the following formula: AA:AA=P/PO. The data were expressed by mean±standard deviation (SD). The t-test was adopted for statistical analysis. P<0.05 indicated a significant difference. All data were analyzed by software.

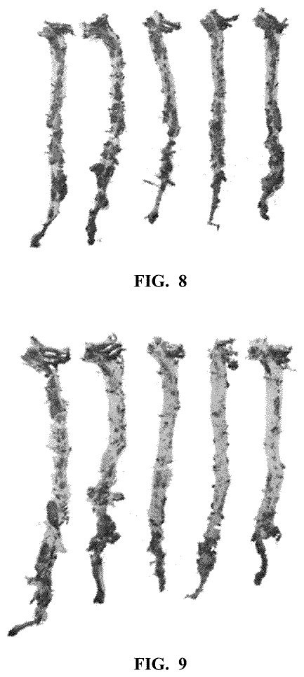

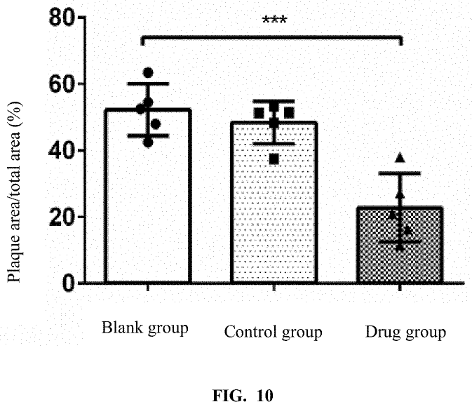

Grouping and administration regimen: Blank group: Each of the mice was intraperitoneally injected with 0.2 mL of NS every two days. Control group: Each of the mice was intraperitoneally injected with a RAPA solution diluted with NS to a desired concentration (administration amount: 1.5 mg/kg) every two days. Drug group: Each of the mice was intraperitoneally injected with the RAPA composition in Example 4 at 1.5 mg/kg every two days. The administration was conducted continuously for 3 months, during which state changes in mice were observed. After the administration was complete, the mice were anesthetized, blood was collected and tested for blood lipid level and some inflammatory factors, and blood vessels were collected to observe the plaque. The effects of AS on the blank group, the control group, and the drug group are shown in FIG. 7 to FIG. 9, respectively, and the results showed that, after the administration, vascular plaques were significantly reduced. As shown in FIG. 10, the plaque area/total vascular area for each group was as follows: blank group: 52.18% 3.497%, control group: 48.33%±2.851%, and drug group: 28.32%±5.461% (p<0.05).

Corresponding changes and variations may be made by those skilled in the art according to the technical solutions and concepts described above, and all these changes and variations should fall within the protection scope of the claims of the present disclosure.

What is claimed is:

1. A rapamycin (RAPA) composition, comprising the following active ingredients in parts by weight:

| | |
|---|---|
| RAPA | 1 to 10 parts; |
| a polymer carrier | 0.5 to 20 parts; and |
| a lymphatic target | 0.1 to 1 part; |

wherein the lymphatic target is at least one selected from the group consisting of sodium hyaluronate (SH), an aptamer, and an antibody;
a specific binding capacity of the aptamer to lymphatic endothelial cells (LECs) is higher than or equal to 50%; and
the antibody is at least one selected from the group consisting of a lymphatic vessel endothelial hyaluronic acid receptor antibody and a human recombinant Prox protein antibody.

2. The RAPA composition according to claim 1, wherein the polymer carrier is at least one selected from the group consisting of polyethylene glycol (PEG), polylactic-co-glycolic acid (PLGA), polyethyleneoxide (PEO), polyvinylpyrrolidone (PVP), polypropylene (PP), polyamino acid (PAA), polysorbate, a polyoxyethylene fatty acid, a methoxypolyethylene glycol (mPEG) block copolymer, and methoxypolyethylene glycol-polylactide (mPEG-PLA).

3. The RAPA composition according to claim 1, wherein an overlap rate between a nucleotide sequence of the aptamer and one of sequences shown in SEQ ID NOs: 1-10 is higher than or equal to 50%.

4. The RAPA composition according to claim 1, wherein the RAPA composition further comprises a phospholipid; and the phospholipid is at least one selected from the group consisting of lecithin, cephalin, phosphatidylserine, phosphatidylglycerol (PG), phosphatidylinositol (PI), sphingomyelin, diphosphatidylglycerol (DPG), dipalmitoylphosphatidylcholine (DPPC), dioleoylphosphatidylethanolamine (DOPE), and distearoylphosphatidylethanolamine (DSPE).

5. The RAPA composition according to claim 1, wherein the RAPA composition further comprises cholesterol.

6. A preparation method of a RAPA composition, comprising:
preparing an organic phase solution by adding RAPA to an organic phase solvent to obtain the organic phase solution;
preparing an emulsion by adding a polymer carrier to an aqueous phase solvent and adding the organic phase solution dropwise to the aqueous phase solvent to obtain the emulsion; and
conducting a homogenization by adding a lymphatic target to the emulsion, mixing, and homogenizing to obtain the RAPA composition.

7. The preparation method of the RAPA composition according to claim 6, wherein in a preparation of the organic phase solution, the organic phase solvent is at least one selected from the group consisting of absolute ethanol, dichloromethane (DCM), tertiary butyl alcohol (TBA), acetone, and methanol.

8. The preparation method of the RAPA composition according to claim 6, wherein in a preparation of the emulsion, a mixing is achieved by stirring for 30 min to 3 h at room temperature and a stirring speed of 300 rpm to 1,200 rpm.

9. The preparation method of the RAPA composition according to claim 6, further comprising:
conducting a lyophilization by adding a lyophilization protective agent to the RAPA composition to obtain a first resulting mixture, filtering the first resulting mixture through a microporous filter membrane for a sterilization to obtain a second resulting mixture, and lyophilizing the second resulting mixture.

10. The preparation method of the RAPA composition according to claim 9, wherein in the lyophilization, the lyophilization protective agent is added at an amount of 5 to 20 g per 100 mL of the RAPA composition.


SEQUENCE LISTING (10 sequences)

SEQ ID NO: 1 - DNA, 81 bp, Artificial Sequence
  The sequence is synthesized
cccctgcaggtgattttgctcaagttgctcgtcgcgatacgtttggttggtgtggttggcagtatcgctaatcaggcggat

SEQ ID NO: 2 - DNA, 81 bp, Artificial Sequence
  The sequence is synthesized
cccctgcaggtgattttgctcaagttgcttgtcgcgatacgtttggttggtgtggttggcagtatcgctaatcaggcggat

SEQ ID NO: 3 - DNA, 81 bp, Artificial Sequence
  The sequence is synthesized
cccctgcaggtgattttgctcaagttgctcatcgcgatacgtttggttggtgtggttggcagtatcgctaatcaggcggat

SEQ ID NO: 4 - DNA, 81 bp, Artificial Sequence
  The sequence is synthesized
cccctgcaggtgattttgctcaagttacttgtcgcgatacgtttggttggtgtggttggcagtatcgctaatcaggcggat

SEQ ID NO: 5 - DNA, 81 bp, Artificial Sequence
  The sequence is synthesized
cccctgcaggtgattttgctcaagttgctcgtcgcgatacgtttggttggtgtggttgggagtatcgctaatcaggcggat

SEQ ID NO: 6 - DNA, 81 bp, Artificial Sequence
  The sequence is synthesized
cccctgcaggtgattttgctcaagttgcttgtcgcgatacgttcggttggtgtggttggcagtatcgctaatcaggcggat

SEQ ID NO: 7 - DNA, 81 bp, Artificial Sequence
  The sequence is synthesized
cccctgcaggtgattttgctcaagttgttcgtcgcgatacgtttggttggtgtggttggcagtatcgctaatcaggcggat

SEQ ID NO: 8 - DNA, 81 bp, Artificial Sequence
  The sequence is synthesized
cccctgcaggtgattttgctcaagttgctcgttgcgatacgtttggttggtgtggttggcagtatcgctaatcaggcggat

SEQ ID NO: 9 - DNA, 81 bp, Artificial Sequence
  The sequence is synthesized
cccctgcaggtgattttgctcaagttgcttgccgcgatacgtttggttggtgtggttggcagtatcgctaatcaggcggat

SEQ ID NO: 10 - DNA, 81 bp, Artificial Sequence
  The sequence is synthesized
cccctgcaggtgattttgctcaagttgctcatcgcgatacggttggttggtgtggttggcagtatcgctaatcaggcggat